(19) United States
(12) Patent Application Publication
(10) Pub. No.: US 2026/0144957 A1
(43) Pub. Date: May 28, 2026

(54) WEARABLE DEVICE COMBINING LIGHT, INFRARED LIGHT AND SOUND THERAPY

(71) Applicant: Dmitri Leonov, Bryn Mawr, PA (US)

(72) Inventor: Dmitri Leonov, Bryn Mawr, PA (US)

(21) Appl. No.: 19/401,457

(22) Filed: Nov. 26, 2025

Related U.S. Application Data
(60) Provisional application No. 63/725,102, filed on Nov. 26, 2024.

(51) Int. Cl. A61M 21/02; A61M 21/00

(52) U.S. Cl. CPC A61M 21/02; A61M 2021/0022; A61M 2021/0027; A61M 2021/0044; A61M 2205/052; A61M 2205/3576; A61M 2205/502; A61M 2205/8206; A61M 2209/088

(57) ABSTRACT

A therapeutic wearable device is disclosed that uses a plurality of different frequencies from the electromagnetic spectrum including frequencies from the visible region, the IR region, the radio region and optionally low frequency vibrational therapy. The therapeutic wearable device can be used to treat any of a plurality of conditions such as depression, seasonal affective disorder, sleep disorders, circadian rhythm disorders, psoriasis, eczema, acne, and/or cancer. The therapeutic wearable device can be incorporated into wearable items such as bracelets, necklaces or garments of clothing.

### CLAIM OF PRIORITY

The present application claims priority under 35 USC 119(e) to US Provisional Application No. 63/725,102, filed on Nov. 26, 2024, the entire contents of which are incorporated by reference in its entirety.

### TECHNICAL FIELD OF THE INVENTION

The present invention relates to the fields of therapeutic wearable devices and frequency medicine. Specifically, this invention is a multi-functional wearable apparatus combining light therapy, infrared light therapy, and sound therapy with an optional vibrational component. The invention addresses mental, physical, and emotional well-being by applying therapeutic frequencies to various parts of the body, intended to stabilize and enhance an individual's nervous system function and overall health.

### BACKGROUND OF THE INVENTION

Modern lifestyles, characterized by high levels of stress and exposure to artificial environments, often contribute to a destabilization of the human nervous system. This destabilization can manifest in a variety of ways, including increased anxiety, sleep disturbances, emotional imbalance, and/or other physical health concerns. Numerous scientific studies including over 11,000 peer-reviewed publications have demonstrated the efficacy of light therapy, infrared therapy, and sound therapy in promoting physical and emotional health, offering relief for symptoms associated with nervous system dysregulation.

Wearable devices have become increasingly popular as individuals seek convenient ways to support their well-being. However, there is currently no single, compact, wearable device that allows the user to benefit from combined light and sound therapies effectively. While individual devices are available for separate therapies, users must often purchase multiple products to achieve combined benefits, which is costly and inconvenient. Furthermore, existing devices lack versatility, as they are often limited to specific body parts and cannot be worn as an accessory.

It is with these drawbacks in mind that the present invention was developed.

### SUMMARY OF THE INVENTION

The present invention addresses the multiple challenges discussed above. One object of the present invention is creating a single, adaptable device that: a) combines therapeutic frequencies of light, infrared light, and sound in a compact, wearable form, b) offers adjustable frequencies for tailored therapeutic effects, c) is portable, allowing users to receive therapeutic benefits regardless of location or activity, d) addresses the growing concern of electromagnetic field exposure.

In an embodiment, the present invention relates to a wearable therapeutic apparatus designed to combine light, infrared light, and sound therapy. Additionally, the device offers a subtle vibrational component, amplifying its therapeutic potential by activating embedded crystals. In a variation, this device is designed to be worn as a necklace or applied to any part of the body. Alternatively, it can be placed near the body, such as on a bedside table during sleep, to provide continuous therapeutic effects.

Accordingly, one advantage of the present device is that because it can be placed on a bedside table during rest is that the individual that requires the light and sound therapy (and optionally the vibrational therapy) can benefit from the advantageous effects of the device during sleep. The individual may not even be aware of the beneficial effects of the device until the individual wakes up and feels remarkable.

Light and Infrared Light Sources: The device includes light and infrared light sources that can be rotated through specific frequencies known to positively impact health and well-being. The light frequencies are configurable, allowing users to select a specific frequency to achieve a desired therapeutic effect or to benefit from alternating frequencies. In one embodiment, the device may be programmable so that different frequencies can be attained by the device by the program that operates the device.

Sound Therapy through a Transducer: The device incorporates a transducer that emits therapeutic sound frequencies. This transducer also generates a subtle vibration in tandem with the sound, which enhances the therapeutic impact by introducing gentle tactile stimulation. This dual-action feature (sound plus vibration) aims to support the user's mental and physical relaxation.

Optional Vibration Component: An optional vibration component is included in the design to expand the therapeutic effect further. This vibration feature works in concert with the other modalities to provide an additional layer of therapy, with adjustable frequency settings for customizable vibration intensities. This component amplifies the effects of the device and, when used in conjunction with the crystals, can activate the crystals'properties for an enhanced user experience.

Embedded Crystals: The device contains two crystals: shungite and clear quartz. Shungite, in combination with copper, helps neutralize electromagnetic fields (EMFs) that users may be exposed to daily. Clear quartz serves as an amplifier of the therapeutic frequencies produced by the device, augmenting the effect of the light, sound, and vibration therapies. When the vibration component is activated, it stimulates the crystals, thereby enhancing their beneficial properties.

Power Supply: The device is powered by a rechargeable battery and includes a USB charging port. This battery-operated functionality allows users to experience uninterrupted therapy throughout the day, without needing a wired power source.

The invention offers multiple modes, including a rotation mode and a fixed mode. The rotation mode cycles through various therapeutic frequencies, allowing the user to benefit from a range of frequencies known to support health and wellness. Alternatively, users can set the device to a specific frequency mode, selecting the exact light, infrared, sound, or vibration frequency that aligns with their personal needs.

The wearable design allows users to easily apply it to different areas of the body or wear it around the neck for convenient use throughout the day. This versatility enables it to be used while on the move or during restful periods, such as sleep.

The present invention's advantages and features are described herein in detail to enable someone with ordinary skill in the art to understand how to practice and how to make the present invention.

### BRIEF DESCRIPTION OF THE SEVERAL VIEWS OF THE DRAWINGS

The figures may not be drawn to scale to ensure better clarity and understanding of the different elements and embodiments of the present invention. Also, commonly understood elements in the industry may be omitted to provide a clearer view of the various embodiments of the present invention.

FIG. 1 is an illustrative representation of a wearable device combining light, infrared light and sound therapy, and optionally vibrational in front view, in accordance with embodiments of the present invention;

FIG. 2 is an illustrative representation of a wearable device combining light, infrared light and sound therapy, in rear view, in accordance with embodiments of the present invention.

FIG. 3 is an illustrative representation of a wearable device combining light, infrared light and sound therapy, showing a schematic, in accordance with embodiments of the present invention;

FIG. 4 is an illustrative representation of a wearable device combining light, infrared light and sound therapy, showing an exploded view, in accordance with embodiments of the present invention.

FIG. 5 shows the treatment of vibrational data wherein it is decoded from rising and falling edges of the vibration pattern using the Manchester E. G. Thomas code.

FIG. 6 shows the data sequence parameters for the device of the present invention including how coding can be written to allow the device to operate.

FIG. 7 shows the parameters for a user interface for a device of the present invention.

### DETAILED DESCRIPTION OF THE INVENTION

The detailed description provided herein is solely intended to serve as an example and is not intended to limit the described embodiments or their applications and uses. The terms “exemplary” or “illustrative” used in this document mean “serving as an example, instance, or illustration,” and do not imply any preferred or advantageous implementation over others. The implementations described in this document are provided as examples to enable those skilled in the art to make or use the embodiments of the disclosure, without limiting the scope of the disclosure. No expressed or implied theory presented in the preceding technical field, background, brief summary, or the following detailed description should be considered binding. The specific devices and processes illustrated in the attached drawings and described in the following specification are exemplary embodiments of the inventive concepts, and therefore, any characteristics, should not be considered limiting, unless expressly stated otherwise.

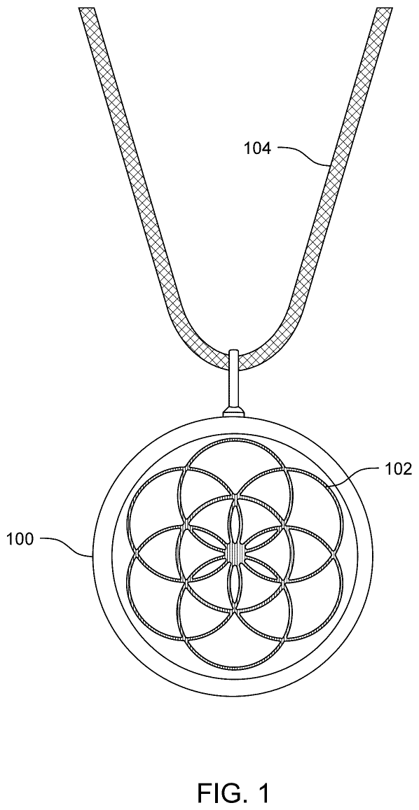

FIG. 1 is an illustrative representation of a wearable device combining light, infrared light and sound therapy, in front view, in accordance with embodiments of the present invention. The invention 100 is shown in front view. The casing/enclosure 102 is copper, plated with 24K gold or platinum. In this instance, the invention is worn as a necklace 104.

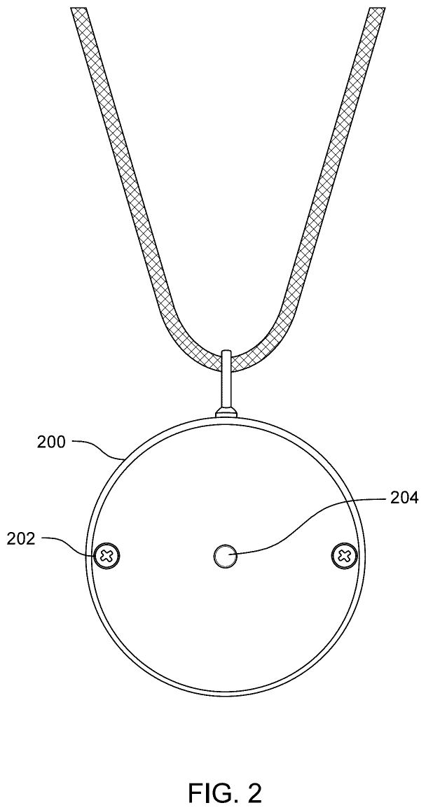

FIG. 2 is an illustrative representation of a wearable device combining light, infrared light and sound therapy, in rear view, in accordance with embodiments of the present invention. The invention 200 is shown in rear view. The casing/enclosure is assembled with screws 202. An opening for emitting diffused light 204. In this instance, the invention is worn as a necklace 206.

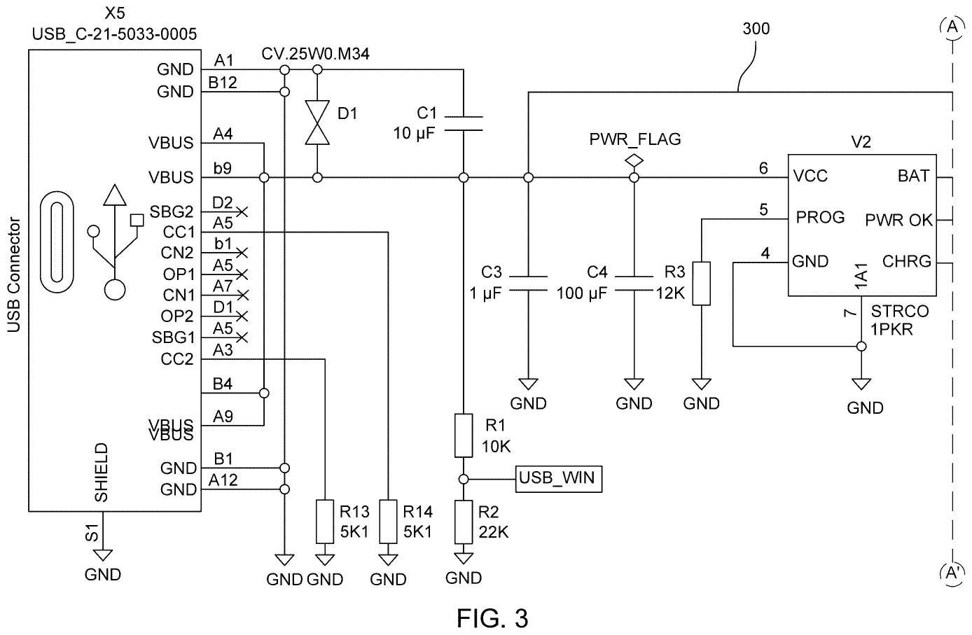

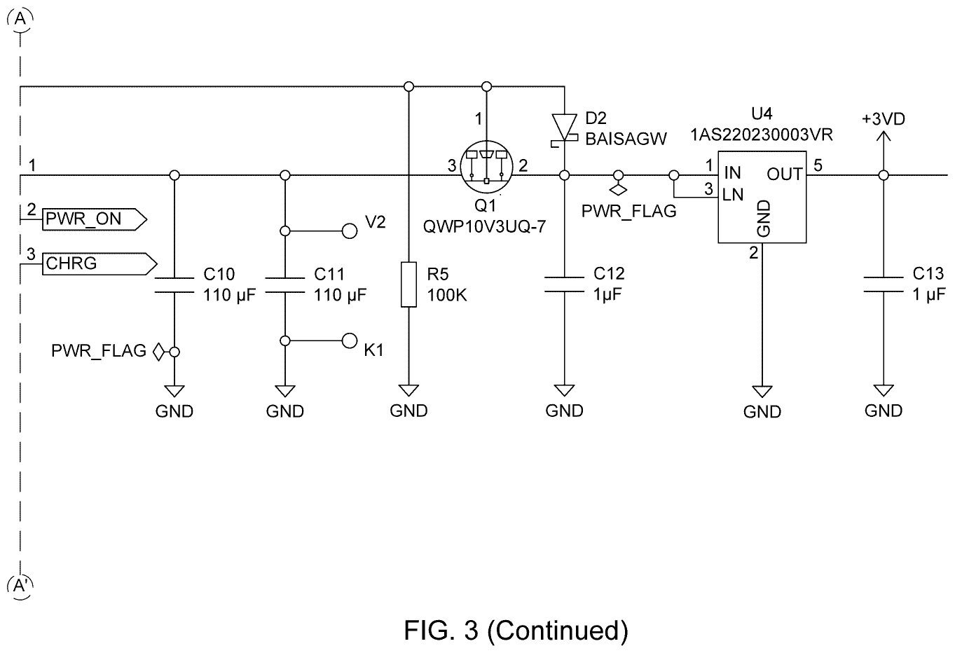

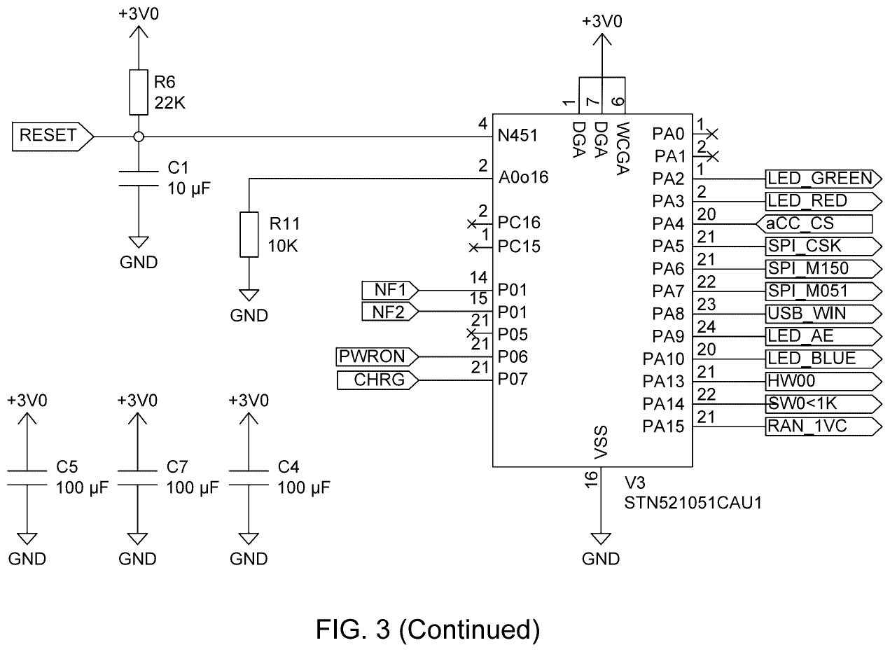

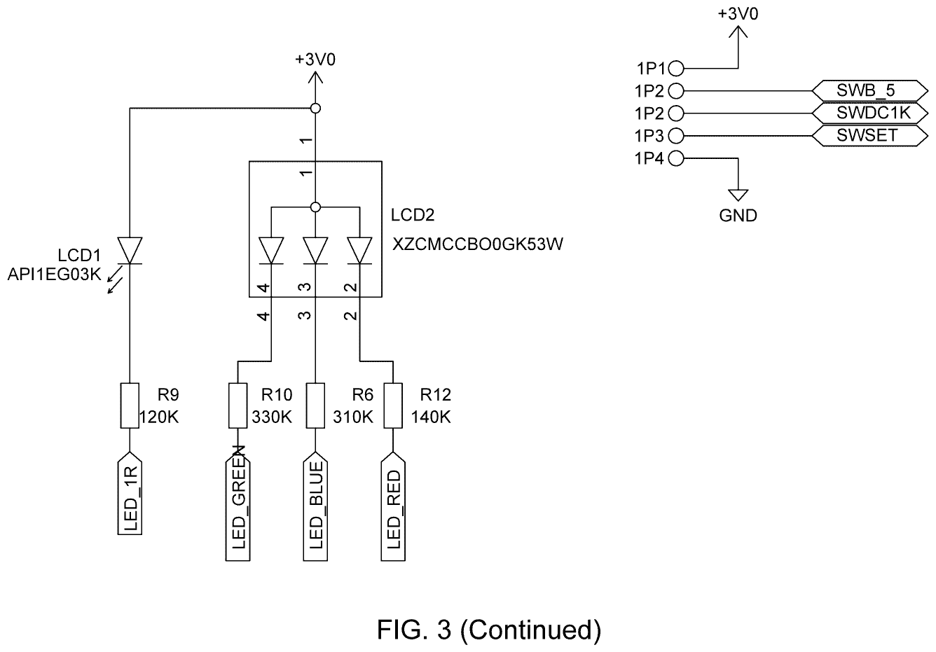

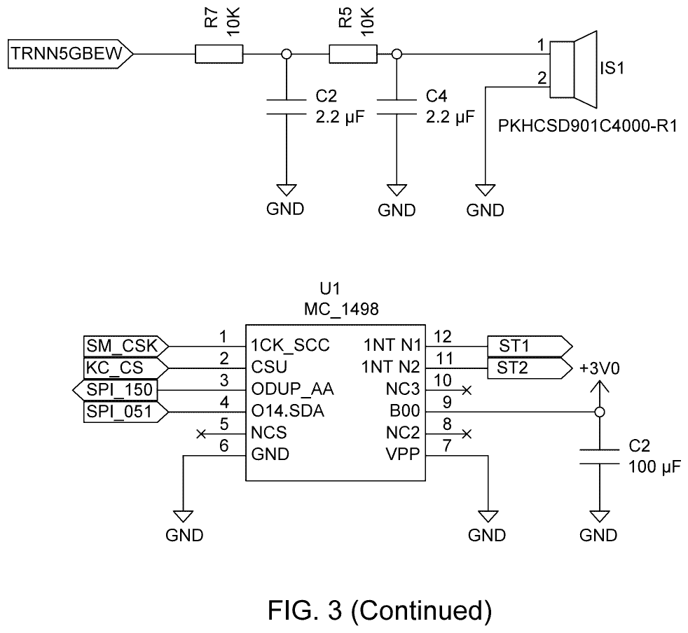

FIG. 3 is an illustrative representation of a wearable device combining light, infrared light and sound therapy, showing a schematic, in accordance with embodiments of the present invention. D1 is a detailed schematic 300 of the invention.

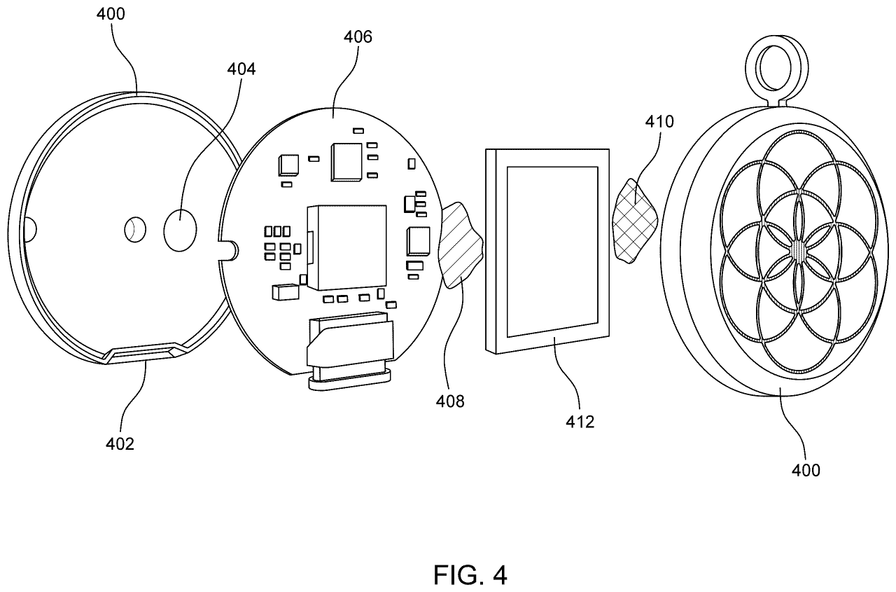

FIG. 4 is an illustrative representation of a wearable device combining light, infrared light and sound therapy, showing an exploded view, in accordance with embodiments of the present invention. The invention is shown in an exploded view. The casing/enclosure 400 is copper, plated with 24K gold or platinum. A charging port 402 is in the base. Nanotechnology light diffusing prism 404. Electronic circuit board 406. Shungite crystal 408. Clear quartz crystal 410. Battery 412. The invention may deliver light therapy, sound therapy, vibration therapy, crystal therapy, or any other variations known in the art.

The present invention addresses the multiple challenges discussed above. One object of the present invention is creating a single, adaptable device that: a) combines therapeutic frequencies of light, infrared light, and sound in a compact, wearable form, b) offers adjustable frequencies for tailored therapeutic effects, c) is portable, allowing users to receive therapeutic benefits regardless of location or activity, d) addresses the growing concern of electromagnetic field exposure.

In an embodiment, the present invention relates to a wearable therapeutic apparatus designed to combine light, infrared light, and sound therapy. Additionally, in one embodiment, the device offers a subtle vibrational component, amplifying its therapeutic potential by activating embedded crystals. In a variation, this device is designed to be worn as a necklace or applied to any part of the body. Alternatively, it can be placed near the body, such as on a bedside table during sleep, to provide continuous therapeutic effects.

Accordingly, one advantage of the present device is that because it can be placed on a bedside table during rest is that the individual that requires the light and sound therapy (and optionally the vibrational therapy) can benefit from the advantageous effects of the device during sleep. The individual may not even be aware of the beneficial effects of the device until the individual wakes up and feels remarkable.

Light and Infrared Light Sources: In one embodiment, the device includes light and infrared light sources that can be rotated through specific frequencies known to positively impact health and well-being. The light frequencies are configurable, allowing users to select a specific frequency to achieve a desired therapeutic effect or to benefit from alternating frequencies. In one embodiment, the device may be programmable so that different frequencies can be attained by the device by the program that operates the device.

Sound Therapy through a Transducer: In one embodime, the device incorporates a transducer that emits therapeutic sound frequencies. This transducer optionally also generates a subtle vibration in tandem with the sound, which enhances the therapeutic impact by introducing gentle tactile stimulation. In one variation, this dual-action feature (sound plus vibration) aims to support the user's mental and physical relaxation.

Optional Vibration Component: In an embodiment, an optional vibration component is included in the design to expand the therapeutic effect further. This vibration feature works in concert with the other modalities to provide an additional layer of therapy, with adjustable frequency settings for customizable vibration intensities. This component amplifies the effects of the device and, when used in conjunction with the crystals, can activate the crystals'properties for an enhanced user experience.

Embedded Crystals: In an embodiment, the device contains two crystals: shungite and clear quartz. Shungite, in combination with copper, helps neutralize electromagnetic fields (EMFs) that users may be exposed to daily. Clear quartz serves as an amplifier of the therapeutic frequencies produced by the device, augmenting the effect of the light, sound, and vibration therapies. When the vibration component is activated, it stimulates the crystals, thereby enhancing their beneficial properties.

Power Supply: In an embodiment, the device is powered by a rechargeable battery and includes a USB charging port. This battery-operated functionality allows users to experience uninterrupted therapy throughout the day, without needing a wired power source.

In an embodiment, the invention offers multiple modes, including a rotation mode and a fixed mode. The rotation mode cycles through various therapeutic frequencies, allowing the user to benefit from a range of frequencies known to support health and wellness. Alternatively, users can set the device to a specific frequency mode, selecting the exact light, infrared, sound, or vibration frequency that aligns with their personal needs. The device of the invention that is designed to cycle through multiple modes in an embodiment can be adjusted and or programmed in a manner that provides optimal results to an individual that is using the device. In a variation, the device may have the ability to read data output from the individual that allows the device to be automatically adjusted, and/or alternatively and/or additionally provides information to the user indicating that an adjustment should be made to obtain optimal results.

In an embodiment, the wearable design allows users to easily apply it to different areas of the body or wear it around the neck for convenient use throughout the day. This versatility enables it to be used while on the move or during restful periods, such as sleep.

The present invention's advantages and features are described herein in detail to enable someone with ordinary skill in the art to understand how to practice and how to make the present invention.

In an embodiment, IR radiation may be generated by heating materials with an electric current (e.g., using non-luminous emitters) or by heating a filament in a bulb (e.g., a tungsten containing filament) or the use of similar luminous generators. Other methods, such as the use of bioceramic nanoparticle materials or the use of moxa sticks may be used to generate IR radiation.

In an embodiment, visible radiation may be generated by passing an electric current through semiconductor materials like light-emitting diodes (LEDs). Because incandescent bulbs are known to generate a wide frequency of irradiation, light bulbs that use materials such as tungsten containing filaments may also be used to generate visible light. Other methodologies including the use of xenon lamps, which may be used to generate irradiation in the visible wavelength region of the electromagnetic spectrum.

In an embodiment, vibrational radiation may be generated by the use of electromagnetism using the piezoelectric effect (i.e., the conversion of electrical energy into mechanical or electromagnetic waves). In a variation, an electrical signal may be passed through crystals such as quartz inside a probe or transducer. The electrical signals cause the quartz to change shape (e.g., vibrate) producing high-frequency waves at a frequency above what a typical human can hear. The micro-vibrations can be used as the source to generate thermal heat or produce microscopic bubbles that can influence the activity of a biological tissue and/or cells that is subject to this irradiation. Alternatively, vibroacoustic technology can be employed that uses specialized speakers or transducers to generate very low frequency sound waves. The movement of the speaker cone can be used in combination with a mat or a rod that generates physical vibrations that can be used by the device of the present invention.

In an embodiment, the present device may use the transmission of information, such as a sound frequency, to the device, wherein the device is capable of detecting any type of translatory motion. The device may be in communication with a smartphone. Optionally, an application (i.e., an app) is used on the smart phone, the app designed and configured to provide useful information to a user that wears the device wherein the device transmits this useful information to the user. For example, if vibrations are generated by a smartphone's vibration generator, they may be detected as repetitive events. In order to detect a continuous vibration stroke, the device may use a debouncing algorithm, so that the start and the end of a vibration stroke are translated into rising and falling edge events.

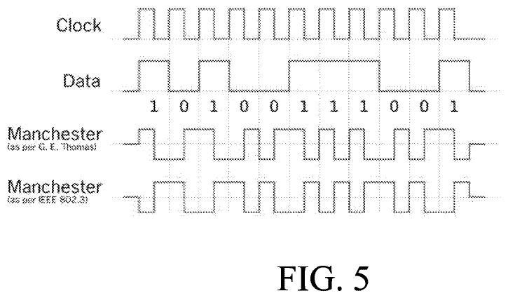

Vibration patterns can be detected. For example, data will be decoded from rising and falling edges of the vibration pattern, using a Manchester E. G. Thomas code as shown in FIG. 5. (figure taken from https://en.wikipedia.org/wiki/Manchester code visited September 20, 2025). Manchester code (also known as phase encoding, or PE) is a line code in which the encoding of each data bit is either low then high, or high then low, for equal time. It is a self-clocking signal with no DC component. Consequently, electrical connections using a Manchester code are easily galvanically isolated. The advantage of Manchester coding is in data transmission, which allows for self-clocking, error detection, and the fact that there is no direct current component. Moreover, another benefit is that it ensures reliable and accurate delivery of an intended frequency and duration of vibration, which is important for delivering a consistent therapeutic experience using the vibrational frequency therapy of the present invention.

In an embodiment, a high frequency generator may generate an electric current that is sent to a transducer containing a piezoelectric crystal. The vibration of crystal causes sound waves to be generated that are able to travel into the body.

In an embodiment, the device of the present invention may be able to generate a wide array of different frequencies from the electromagnetic spectrum using the above mentioned techniques. In one variation, an electrical signal may pass through any of a number of different media (such as those discussed above) in order to generate a plurality of different frequencies from the electromagnetic spectrum. In one variation, an oscillator circuit may generate a repetitive waveform, which can be shaped by one or more modulators and through one or more amplifiers. The resulting electrical signal can be modulated in a manner that elicits the various frequencies in the electromagnetic spectrum eluded to above.

In an embodiment, an electrical signal can be generated from a concentration gradient in a device, which is achieved by using a selective membrane or channel that allows specific ions to move down the concentration gradient, creating a separation of charge that results in an electrical potential or voltage. Nanofluidic devices, triboiontronic generators, microfluidic systems and/or the use of polysaccharide films may allow this concentration and electrical signal to be generated.

In an embodiment, a power source, the requisite conductive paths such as electrical wire (e.g., aluminum or copper), and an active component to create the signal such as an oscillator, which may be a part of a larger integrated circuit, may all be present in the devices of the present invention. The integrated circuit may contain or more of transistors, resistors, capacitors, semiconductors, diodes, logic gates, microcontrollers, and/or microprocessors. Other electrical components that may optionally be present in the device of the present invention include but are not limited to batteries, switches, circuit boards, relays, transformers, potentiometers, inductors, thermistors, thyristors, voltage regulators, fuses, and/or oscillators.

The power source may comprise one of more of a battery, a fuel cell, a power supply, a photovoltaic device, a thermoelectric generator, an electrical generator, a piezoelectric generator, and a Van de Grraff generator.

The device of the present invention may be worn as a necklace, wristband, be present in a pocket, may be incorporated into a garment (e.g., in hats such as ski hats, cowboy hats, or other hats, in shirts or jackets, incorporated into pants, shorts, socks, shoes including sandals and flip flops, belts, belt buckles, dresses, or any other garments) or be generated in other variations, including into devices that may be known in the art. In a variation, the device may be in the form of jewelry including but not limited to the bracelet and necklace discussed above, a broach, a pin, earrings, anklets, body jewelry, watches, rings, and other types of jewelry. The device may be aesthetically pleasing and be disguised so that it is not readily apparent that the device is present.

The manufacturing method may make use of injection molding, rotational molding, CNC milling, laser cutting, 3D printing, or any other variations known in the art.

The invention may be formed of copper, gold, silver, platinum, ceramic, silicone, aluminum, stainless steel, polypropylene, nylon, polyester, or any other variations known in the art.

In an embodiment, the device may be in communication with a smartphone and/or a computer. The requisite electronics may be present to allow this communication including but not limited to an antenna, a wifi (i.e., wireless) module, and a microprocessor. The wifi module may comprise one or more of a microcontroller, an RF chipset, a baseband processor for signal processing, power amplifiers, and/or low-noise amplifiers. The RF chipset may comprise one or more of an antenna, resistors, capacitors, inductors, amplifiers, oscillators, mixers, filters, modulators, demodulators, switches, and analog to digital converters and digital to analog converters. Optionally, an application (i.e., an app) is used on the smart phone, the app designed and configured to provide useful information to a user that wears the device wherein the device transmits this useful information to the user. The useful information may be information about the health of the user, recommendations for the therapy that the user should emphasize, information about a schedule that the user should employ when using the device, or other information that may be useful to the user.

In an embodiment, the device of the present invention is able to receive/accept data from the health of the user. For example, the device may be able to use an optical heart sensor to analyze how a user's blood vessels respond to and or correlated with the heart beats of a user. The device in this instance would require a sensor that is able to emit light wherein the light is shined into a section/body part of a user (for example, on a wrist) to measure the light absorbed versus that reflected and use an algorithm to measure blood flow relative to the frequency of heart beats. The device may then be able to notify the user of the likelihood of high blood pressure based upon data stored in the algorithm. The algorithm may also be a learning algorithm that is able to individualize data and notify a user of specific deviations from baseline data from a user.

In an embodiment, the device of the present invention notifies a user of the device via smart phone when the health of the user requires it, for example, when a change in the health of a user is detected.

In an embodiment, the present invention relates to a therapeutic wearable device for healing an individual in need thereof, the wearable device comprising a power supply wherein the therapeutic wearable device is configured to supply one or more of visible light frequency therapy, infrared radiation frequency therapy, sound radiation frequency therapy and vibrational frequency therapy. The device may comprise visible light frequency therapy, infrared radiation frequency therapy, sound radiation frequency therapy and optionally vibrational frequency therapy or any variation of these therapies. In a variation, the therapeutic wearable device is configured to be integrated into a garment, a necklace, or a bracelet. In a variation, the garment is a hat, a shirt, a sock, a dress, a shoe, a jacket, pants, or shorts. In a variation, the therapeutic wearable device comprises one or more of a transistor, a diode, a resistor, a capacitor, a semiconductor, a logic gate, a microcontroller, and/or a microprocessor.

In an embodiment, the therapeutic wearable device further comprises one or more of a battery, a switch, a circuit board, a relay, a transformer, a potentiometer, an inductor, a thermistor, a thyristor, a voltage regulator, a fuse, and/or an oscillator. In a variation, the therapeutic wearable device further comprises at least one crystal. In a variation, the crystal is a quartz crystal or a shungite crystal. In a variation, the therapeutic wearable device further comprises an electric current that passes through a light emitting diode, a crystal, and/or a transducer.

In an embodiment, the electric current passes through all of the light emitting diode, the crystal, and the transducer thereby generating a wide array of frequencies from the electromagnetic spectrum. In a variation, the therapeutic wearable device is configured to be able to communicate with a smartphone. In a variation, the communication with the smart phone is achieved by using the electronic components discussed herein. In a variation, the smartphone comprises an application that is configured to provide information to a user from the therapeutic wearable device, and the application allows for input that is configured to provide instructions to the therapeutic wearable device. In a variation, the vibrational frequency therapy is present, and the vibrational frequency therapy comprises data decoded from rising and falling edges of a vibration pattern, using a Manchester E. G. Thomas code. In a variation, the use of Manchester E. G. Thomas code provides the benefits discussed herein.

In an embodiment, the present invention relates to a method of providing therapy to a user in need thereof comprising administering therapy from a therapeutic wearable device, said therapeutic wearable device comprising a power supply wherein the therapeutic wearable device is configured to supply one or more of visible light frequency therapy, infrared radiation frequency therapy, sound radiation frequency therapy and vibrational frequency therapy. In a variation, any two of these therapies may be present in the device. In a variation, any three of these therapies may be present in the device.

In a variation of the method, the therapeutic wearable device is part of a bracelet, necklace, or garment. In a variation, the therapeutic wearable device is configured to be able to communicate with a smartphone. In a variation, the therapeutic wearable device is configured to be able to communicate with a smartphone wherein the smartphone comprises an application that is configured to provide information to a user from the therapeutic wearable device, and the application allows for input that is configured to provide instructions to the therapeutic wearable device.

In an embodiment, the present invention relates to a method of treating an individual for depression, seasonal affective disorder, sleep disorders, circadian rhythm disorders, psoriasis, eczema, acne, or cancer comprising administering therapy from a therapeutic wearable device, said therapeutic wearable device comprising a power supply wherein the therapeutic wearable device is configured to supply visible light frequency therapy, infrared radiation frequency therapy, sound radiation frequency therapy and optionally vibrational frequency therapy.

In a variation, the therapeutic wearable device is configured to be able to communicate with a smartphone and wherein the smartphone comprises an application that is configured to provide information to a user from the therapeutic wearable device, and the application allows for input that is configured to provide instructions to the therapeutic wearable device. In a variation, the therapeutic wearable device is part of a bracelet, necklace, or garment. In a variation, the therapeutic wearable device utilizes vibrational frequency therapy.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the present application.

We claim:

1. A therapeutic wearable device for healing an individual in need thereof, the wearable device comprising a power supply wherein the therapeutic wearable device is configured to supply any one or more of visible light frequency therapy, infrared radiation frequency therapy, sound radiation frequency therapy and vibrational frequency therapy.

2. The therapeutic wearable device of claim 1, wherein the therapeutic wearable device is configured to be integrated into a garment, a necklace, or a bracelet.

3. The therapeutic wearable device of claim 2, wherein the garment is a hat, a shirt, a sock, a dress, a shoe, a jacket, pants, or shorts.

4. The therapeutic wearable device of claim 1, wherein the therapeutic wearable device comprises one or more of a transistor, a diode, a resistor, a capacitor, a semiconductor, a logic gate, a microcontroller, and/or a microprocessor.

5. The therapeutic wearable device of claim 4, wherein the therapeutic wearable device further comprises one or more of a battery, a switch, a circuit board, a relay, a transformer, a potentiometer, an inductor, a thermistor, a thyristor, a voltage regulator, a fuse, and/or an oscillator.

6. The therapeutic wearable device of claim 1, wherein the therapeutic wearable device further comprises at least one crystal.

7. The therapeutic wearable device of claim 6, wherein the crystal is a quartz crystal or a shungite crystal.

8. The therapeutic wearable device of claim 1, wherein the therapeutic wearable device further comprises an electric current that passes through a light emitting diode, a crystal, and/or a transducer.

9. The therapeutic wearable device of claim 8, wherein the electric current passes through all of the light emitting diode, the crystal, and the transducer.

10. The therapeutic wearable device of claim 8, wherein the therapeutic wearable device is configured to be able to communicate with a smartphone.

11. The therapeutic wearable device of claim 10, wherein the smartphone comprises an application that is configured to provide information to a user from the therapeutic wearable device, and the application allows for input that is configured to provide instructions to the therapeutic wearable device.

12. The therapeutic wearable device of claim 11, wherein vibrational frequency therapy is present, and the vibrational frequency therapy comprises data decoded from rising and falling edges of a vibration pattern, using a Manchester E. G. Thomas code.

13. A method of providing therapy to a user in need thereof comprising administering therapy from a therapeutic wearable device, said therapeutic wearable device comprising a power supply wherein the therapeutic wearable device is configured to supply one or more of visible light frequency therapy, infrared radiation frequency therapy, sound radiation frequency therapy and vibrational frequency therapy.

14. The method of claim 13, wherein the therapeutic wearable device is part of a bracelet, necklace, ring, watch, broach, pin, or garment.

15. The method of claim 14, wherein the therapeutic wearable device is configured to be able to communicate with a smartphone.

16. The method of claim 14, wherein the therapeutic wearable device is configured to be able to communicate with a smartphone and wherein the smartphone comprises an application that is configured to provide information to a user from the therapeutic wearable device, and the application allows for input that is configured to provide instructions to the therapeutic wearable device.

17. A method of treating an individual for depression, seasonal affective disorder, sleep disorders, circadian rhythm disorders, psoriasis, eczema, acne, or cancer comprising administering therapy from a therapeutic wearable device, said therapeutic wearable device comprising a power supply wherein the therapeutic wearable device is configured to supply one or more of visible light frequency therapy, infrared radiation frequency therapy, sound radiation frequency therapy and vibrational frequency therapy.

18. The method of claim 17, wherein the therapeutic wearable device is configured to be able to communicate with a smartphone and wherein the smartphone comprises an application that is configured to provide information to a user from the therapeutic wearable device, and the application allows for input that is configured to provide instructions to the therapeutic wearable device.

19. The method of claim 17, wherein therapeutic wearable device is part of a bracelet, necklace, or garment.

20. The method of claim 17, wherein the therapeutic wearable device utilizes vibrational frequency therapy.
